(19) United States
(12) Patent Application Publication
(10) Pub. No.: US 2019/0350918 A1
(43) Pub. Date: Nov. 21, 2019

(54) PPARy AGONIST FOR TREATMENT OF HUNTINGTON'S DISEASE

(71) Applicant: Coherus Biosciences, Inc., Redwood City, CA (US)

(72) Inventors: Barbara Finck, San Francisco, CA (US); Lawrence Steinman, Stanford, CA (US)

(21) Appl. No.: 16/476,979

(22) PCT Filed: Jan. 18, 2018
(86) PCT No.: PCT/US18/14240

§ 371 Date: Jul. 10, 2019

Related U.S. Application Data
(60) Provisional application No. 62/447,741, filed on Jan. 18, 2017.

(51) Int. Cl. A61K 31/47; A61P 25/28

(52) U.S. Cl. CPC A61K 31/47; A61P 25/28

(57) ABSTRACT

Methods of treatment of Huntington's disease or its symptoms, with PPARγ agonists, and in particular, the compound of formula (I) known as INT131:

### FIELD OF THE INVENTION

The present invention relates to methods of treatment of Huntington's disease.

### BACKGROUND OF THE INVENTION

Huntington's disease is a fatal genetic disease caused by a defect the gene HTT. Normally, the gene has between 10-35 CAG repeats. In Huntington's disease, the gene has 36 or more CAG repeats. This results in an abnormal huntingtin protein having a repeat of 36 or more glutamine residues. These mutant proteins aggregate in the brain (e.g. the cortex and striatum) causing neuronal degradation. Brain cells lose function and die over the course of the disease, which results in the afflicted person developing phenotypic symptoms of the disease and eventually dying.

Symptoms of Huntington's disease usually first appear between the ages of 30 to 50 and worsen over about 10 to 30 years until the person dies. When onset begins before the age of twenty, the condition is referred to as juvenile Huntington's disease and death usually occurs with 10 years. People with Huntington's disease commonly die from pneumonia, heart failure, or other complications caused by the loss of functional brain cells and motor function (e.g. the ability to swallow).

Signs and symptoms of Huntington's disease are categorized into movement, cognitive, and psychiatric disorders. Voluntary and involuntary movement can be impaired in Huntington's disease and include: involuntary jerking or writhing movements (chorea), muscle problems, such as rigidity or muscle contracture (dystonia), slow or abnormal eye movements, impaired gait, posture and balance, difficulty with the physical production of speech or swallowing. Cognitive impairments include: difficulty organizing, prioritizing or focusing on tasks, lack of flexibility or the tendency to get stuck on a thought, behavior or action (perseveration), lack of impulse control that can result in outbursts, acting without thinking and sexual promiscuity, lack of awareness of one's own behaviors and abilities, slowness in processing thoughts or “finding” words, difficulty in learning new information. Psychiatric disorders include: depression, feelings of irritability, sadness or apathy, social withdrawal, insomnia, fatigue and loss of energy, frequent thoughts of death, dying or suicide, obsessive-compulsive disorder, mania, bipolar disorder. Additionally, weight loss is a common symptom of Huntington's disease, especially as the disease progresses.

Symptoms in youth with juvenile Huntington's disease may differ in onset and progression from the disease in adults. Early in the course of disease symptoms include behavioral changes such as: loss of previously learned academic or physical skills, rapid, significant drop in overall school performance, and behavioral problems; and physical changes such as: contracted and rigid muscles that affect gait (especially in young children), changes in fine motor skills that might be noticeable in skills such as handwriting, tremors or slight involuntary movements, and seizures.

Progression of Huntington's disease is divided into three stages: early, middle, and late stage. In early stage, a person with Huntington's disease may suffer small changes in coordination, some chorea, trouble thinking through problems, and often irritable moods or depression. Generally, at this stage, a person is less able to work at a level they are accustomed to and are less functional in regular activities.

In the middle stage, ordinary activities become increasingly harder to do. Movement disorders are more pronounced and speaking and swallowing become more difficult. Medication for chorea is often prescribed to control involuntary movement. Control of voluntary movement declines as do thinking and reasoning abilities. To these symptoms occupational and physical therapies may be needed to help retain function.

Late stage Huntington's disease is characterized by total dependency on others. Physical disabilities may render the person unable to walk, talk, or swallow. As such, choking is concern. A person with Huntington's disease will typically die from a complication related to these disabilities, such as choking or an infection.

While there are therapies available to lessen some symptoms of Huntington's disease, none protect the brain or slow the deterioration of nerve cells. Also, as the disease progresses, and symptoms worsen, medications may fail to adequately lessen the symptoms.

Preclinical in vitro and mouse model data indicate that rosiglitazone (Avandia®) and pioglitazone (Actos®), both peroxisome proliferator-activated receptor gamma (PPARγ) full agonists, may benefit patients with Huntington's disease. Despite these data, neither of these compounds have been selected for clinical trials in Huntington's disease.

Notably, there are disadvantages to treating humans with rosiglitazone and pioglitazone. Avandia is only approved for treating patients with type 2 diabetes and increases the risk of: heart failure, cardiovascular events in individuals with heart failure, edema, weight gain, macular edema, bone fractures, decreases in hemoglobin and hemocrit, and other adverse events. Avanida package insert, September 2016. Since only 9-14% of rosiglitazone crosses the blood brain barrier, it may have limited efficacy or require more frequent and higher doses to adequately treat neurological disorders such as Huntington's disease. Like Avandia, Actos is only approved for treating patients with type 2 diabetes. Actos also caries several serious warnings and precautions including: increased risk of fluid retention leading to congestive heart failure, hypoglycemia, sometimes fatal hepatic failure, bladder cancer, edema, bone fractures, macular edema, and other adverse events. Actos package insert, December 2016.

Accordingly, there is need for new safe and effective treatments of Huntington's disease.

### SUMMARY OF THE INVENTION

It has now been discovered that the peroxisome proliferator-activated receptor gamma (PPARγ) agonist INT131 is effective for treating Huntington's disease. The PPARγ is a transcription factor belonging to the steroid/thyroid/retinoid receptor superfamily. To date, PPARγ agonists have been therapeutic agents for disorders such as obesity, diabetes and dyslipidemia.

In one aspect, the present invention provides methods of treating Huntington's disease and symptoms thereof. The methods typically involve administering to a subject in need thereof a therapeutically effective amount of compound INT131 described in U.S. Pat. No. 7,601,841. INT131 is unique among PPARγ agonists in that it exerts potent anti-inflammatory effects in the central nervous system without evidence of systemic immunosuppression and is a selective activator of a highly limited number of PPARγ pathways. Among these INT131-sensitive pathways are metabolic pathways including those pathways regulated by the hormone adiponectin.

As a result of this selective activation, administration of INT131 to patients results in fewer side effects than administration of other PPARγ agonists. For example, INT131 was equally efficacious in reducing HbA1c levels as 45 mg of pioglitazone but subjects taking INT131 experienced less edema, weight gain, and hemodilution than those taking pioglitazone. See, DePaoli, et al. Diabetes Care. 2014 July; 37(7):1918-23. Thus, INT131 can administered to treat Huntington's disease while limiting side effects. Limiting side effects is advantageous as it helps preserve the quality of life for subject taking the medication and results in improved subject compliance with taking medication.

In particular, the invention provides a method of treating Huntington's disease or symptoms thereof in a subject in need thereof comprising administering to the subject a pharmaceutical composition comprising a therapeutically effective amount of a compound of formula (I),

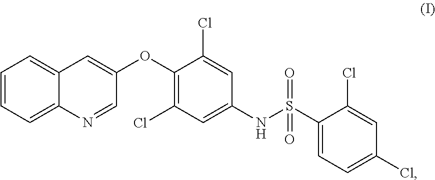 [SMILES: O=S(=O)(Nc1cc(Cl)c(Oc2cnc3ccccc3c2)c(Cl)c1)c1ccc(Cl)cc1Cl]

or a pharmaceutically acceptable salt, prodrug, or isomer thereof.

In one embodiment, the compound of formula (I) (i.e, INT131) is provided in the form of a besylate salt.

In one embodiment, the therapeutically effective amount is from about 0.1 to about 15 mg, more preferably from about 1 to about 10 mg, even more preferably from about 2 to about 6 mg, and most preferably about 3 mg. In another embodiment, the therapeutically effective amount is about 15 mg, about 14 mg, about 13 mg, about 12 mg, about 11 mg, about 10 mg, about 9 mg, about 8 mg, about 7 mg, about 6 mg, about 5 mg, about 4 mg, about 3 mg, about 2 mg, or about 1 mg.

The pharmaceutical compositions used in the methods of the invention may be administered to the subject twice a day, daily, every other day, three times a week, twice a week, weekly, every other week, twice a month, or monthly.

Preferably, the methods of the invention result in increase of the adiponectin level in the subject by at least about 30%, at least about 68%, at least about 175%, or at least about 200%.

### DETAILED DESCRIPTION OF THE INVENTION

In particular, the compound (I),

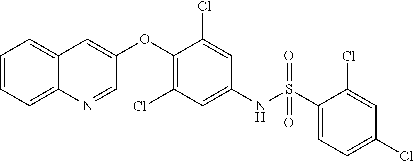 [SMILES: O=S(=O)(Nc1cc(Cl)c(Oc2cnc3ccccc3c2)c(Cl)c1)c1ccc(Cl)cc1Cl]

has been found to be unexpectedly effective for the treatment of Huntington's disease. This compound is also known as INT131 or CHS131.

### Definitions

The terms “treat”, “treating” and “treatment” refer to a method of alleviating or abrogating a disease and/or its attendant symptoms. In another embodiment, treating refers to slowing or halting progression of a disease. In yet another embodiment, treating refers to extending the life of a subject with a disease.

The term “Huntington's disease” refers to the autosomal dominant neurodegenerative disease caused by a CAG trinucleotide expansion in the Huntingtin (Htt) gene. Since Huntington's disease is genetic, the disease is present in a subject who has the mutant gene, whether or not phenotypic signs and symptoms are present.

The term “therapeutically effective amount” refers to that amount of the compound being administered sufficient to treat a disease. In one embodiment, the therapeutically effective amount is sufficient to prevent development of or alleviate to some extent one or more of the symptoms of the condition or disorder being treated.

The term “subject” is defined herein to include animals such as mammals, including but not limited to, primates (e.g., humans), cows, sheep, goats, horses, dogs, cats, rabbits, rats, mice and the like. In preferred embodiments, the subject is a human.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either net or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either net or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isbutyric, oxalic, maleic, malonic, benzoic, succinic, suberic, fumeric mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present inventions contain both basic and acidic functionalities that allow the compounds to be converted into either base or acid addition salts.

The neutral forms of the compounds may be registered by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In additional to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, be bioavailable by oral administration whereas the parent drug is not. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity. Additional examples include peptidyl derivatives of a compound of the invention.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

### Embodiments of the Invention

A new use of a known compound that modulates PPARγ has now been discovered. Specifically, it has been discovered that PPARγ agonists, and in particular, INT131, are effective to treat Huntington's disease.

Thus, in one embodiment, the present invention is directed to a method of treating Huntington's disease or its symptoms in a subject in need thereof comprising administering to the subject a pharmaceutical composition comprising a therapeutically effective amount of INT131 or a pharmaceutically acceptable salt, prodrug, or isomer thereof.

Without wishing to be limited to a particular theory, it is believed that INT131 increases PPARγ activation in brain cells (including activation of elements in the PPARγ pathway), increases adiponectin levels, improves energy metabolism in brain cells, and reduces or prevents aggregation of mutant Huntintin, and therefore, treats Huntington's disease.

Adipose tissue dysfunction was observed in Huntington's disease mouse models. It is detectable at early ages and becomes more pronounced as the disease progresses. Adipocytes acquire a ‘de-differentiated’ phenotype characterized by impaired expression of fat storage genes. These mice exhibit reduced levels of leptin and adiponectin-hormones derived from adipose tissue that regulate food intake and glucose metabolism. Phan et al., Adipose tissue dysfunction tracks disease progression in two Huntington's disease mouse models, Human Molecular Genetics, 2009, Vol. 18, No. 6. Thus, it is now believed that INT131 can treat Huntington's disease by increasing adiponectin levels. INT131 mediated increase in adiponectin increase appetite, increases glucose metabolism, and reduces or prevents weight loss in subjects with Huntington's disease. Recovering impaired glucose metabolism in brain cells improves overall function of the cells and delays onset, or reduces, signs and symptoms of Huntington's disease that result from reduced or dysfunctional glucose metabolism.

Despite data for full PPARγ agonists (e.g. pioglitazone and rosiglitazone) in Huntington's disease, the benefits of INT131 is surprising since it was unknown if the selective PPARγ pathway activation of INT131 would treat Huntington's disease.

Accordingly, it is surprising and unexpected that INT131 treats Huntington's disease.

In one embodiment, INT131 prophylactically treats Huntington's disease. In another embodiment, INT131 prevents or delays the onset of Huntington's disease signs and symptoms. In another embodiment, INT131 reduces the signs and symptoms of Huntington's disease.

In one embodiment, INT131 is neuroprotective in a subject with Huntington's disease. In another embodiment, INT131 treats neuronal degeneration. In another embodiment, INT131 reduces atrophy or degeneration of the brain in subjects with Huntington's disease. In a further embodiment, INT131 reduces atrophy or degradation of the striatum, cortex, hypothalamus, or hippocampus. In another embodiment, INT131 protects the liver in a patient with Huntington's disease.

In one embodiment, INT131 treats weight loss in a subject with Huntington's disease. In further embodiment, INT131 reduces or prevents weight loss. In another embodiment, INT131 increases appetite in a subject with Huntington's disease.

In another embodiment, INT131 treats metabolic dysfunction in a subject with Huntington's disease. In yet a further embodiment, INT131 increases adiponectin levels in a subject with Huntington's disease. In a further embodiment, INT131 reduces adipose tissue dysfunction in a subject with Huntington's disease. In another embodiment, INT131 improves or increases glucose metabolism in a subject with Huntington's disease. In another embodiment, INT131 reduces hyperglycemia in a subject with Huntington's disease.

In another embodiment, the methods of the invention do not result in an increase in adipocytes or adipose tissue.

In another embodiment, the methods of the invention increase glucose metabolism in brain cells.

In another embodiment, the methods of the invention increase glucose metabolism in adipose tissue.

In another embodiment, the methods of the invention reduce the metabolic dysregulation in the subject.

In another embodiment, the methods of the invention reduce insulin resistance in the subject.

In another embodiment, INT131 improves mitochondrial function. In a further embodiment, INT131 improves mitochondrial calcium handling, and mitochondrial trafficking. In yet a further embodiment, INT131 increases the expression of peroxisome proliferator-activated receptor gamma coactivator 1-alpha (PGC-1α) and mitochondrial biogenesis. This results in improved behavior, improved survival (i.e. lifespan) and reduced brain, muscle and brown adipose tissue (BAT) in a subject with Huntington's disease.

In another embodiment, INT131 reduces the aggregation of mutant huntingtin protein, or fragments of mutant huntingtin protein, in a subject with Huntington's disease. In another embodiment, INT131 improves or increases protein degradation in a subject with Huntington's disease. In a further embodiment, INT131 ameliorates the reduction of neuroprotective proteins in the brain. In yet a further embodiment, INT131 reduces the reduction of brain-derived neurotrophic factor and Bcl-2.

In one embodiment, INT131 is in the form of a besylate salt.

In another embodiment, the therapeutically effective amount is from about 0.1 to about 10 milligrams, preferably from about 1 to about 4 milligrams and more preferably from about 2 to about 3 milligrams.

In another embodiment, a composition comprising a therapeutically effective amount of INT131 is administered to a subject in need thereof at an interval that includes, but is not limited to, twice a day, daily, every other day, three times a week, twice a week, weekly, every other week, twice a month, monthly, and every other month.

In another embodiment, a composition comprising a therapeutically effective amount of INT131 is administered orally to a subject. In yet another embodiment, the composition is substantially the same as those disclosed in US Publication 2013-0243865, the disclosure of which is expressly incorporated herein by reference.

In one embodiment, INT-131 is as effective, or more effective, treating Huntington's disease than other therapies. These therapies include therapies approved for treating Huntington's disease and those in development for treating Huntington's disease. These therapies include, but are not limited to, medications to treat movement disorders, medications to treat psychiatric disorders, psychotherapy, speech therapy, physical therapy, and occupational therapy.

Medications to treat movement disorders include, but are not limited to, Tetrabenazine, Antipsychotic drugs, such as haloperidol, chlorpromazine, risperidone, and quetiapine, and other medications such as amantadine, levetiracetam, and, clonazepam.

Medications to treat psychiatric disorders include, but are not limited to, antidepressants such as citalopram, fluoxetine, and sertraline, antipsychotic drugs such as quetiapine, risperidone, and olanzapine, and mood-stabilizing drugs, including anticonvulsants, such as valproate, carbamazepine, and lamotrigine.

Psychotherapy includes, but is not limited to, talk therapy to help a subject manage behavioral problems, depression, and suicidal thoughts.

Speech therapy includes, but is not limited to, improving a subjects ability to speak clearly, and improve function and control of muscles used for eating and swallowing.

Physical therapy includes, but is not limited to, enhancing strength, flexibility, balance and coordination, reducing the risk of falls, and improve posture to lessen the severity of movement problems.

Occupational therapy includes, but is not limited to, use of assistive devices that improve functional abilities such as handrails, and eating and drinking utensils for subjects with diminished motor skills.

In another embodiment, INT-131 is administered to a subject in need thereof in combination with one or more therapies listed herein.

### EXAMPLES

### Example 1: INT131 is a Potent Upregulator of Adiponectin in Patients with Reduced Adiponectin Levels

### Method

A randomized, double-blind, placebo-controlled, 24-week study was conducted in which adiponectin levels were measured. The study had a 2-week lead-in period, a 24-week double-blind treatment period and a 2-week follow up period. 367 subjects with type 2 diabetes (TD2)—a disease in which patient adiponectin levels are reduced—were randomly assigned to receive either 0.5, 1, 2 or 3 milligrams (“mg”) of INT131 besylate, 45 mg of pioglitazone or placebo daily for 24 weeks. To measure adiponectin levels blood was drawn at Weeks 0, 2, 6, 12 and 24.

The results of this study demonstrated that 1, 2, and 3 mg doses of INT131 caused a statistically significant reduction of HbA_{1c} levels as compared to placebo. Further, the study demonstrated that the 2 and 3 mg doses of INT131 reduced HbA_{1c} levels at least as well as 45 mg of pioglitazone, which is an FDA approved treatment for TD2. See, DePaoli, et al. Diabetes Care 2014; 37:1918-1923. Thus, 2 and 3 mg doses of INT131 would be effective in treating TD2.

### Adiponectin Results

At baseline (Week 0) mean adiponectin levels were 1.94 micrograms per milliliter (“μg/mL”). The mean adiponectin levels at baseline and Week 24, and the mean change in adiponectin levels from baseline (Week 0) to Week 24 are disclosed in Table 1, below. The standard deviation for samples tested in each group is listed in (parenthesis). Mean baseline adiponectin values were similar for the treatment groups.

TABLE 1
Changes in Adiponectin Serum Levels

| Mean | | | | | | 45 mg |
|---|---|---|---|---|---|---|
| Adiponectin | | 0.5 mg | 1 mg | 2 mg | 3 mg | Pioglita- |
| (ng/mL) | Placebo | INT131 | INT131 | INT131 | INT131 | zone |
| n | 56 | 56 | 59 | 60 | 60 | 57 |
| Week 0 | 1.85 | 1.73 | 1.87 | 1.87 | 2.00 | 2.32 |
| | (1.153) | (1.190) | (1.217) | (1.098) | (1.215) | (2.185) |
| Week 24 | 1.9 | 2.28 | 3.15 | 5.14 | 5.83 | 5.28 |
| | (1.510) | (1.540) | (2.533) | (3.650) | (4.826) | (3.222) |
| Mean | 0.05 | 0.56 | 1.28 | 3.27 | 3.83 | 2.96 |
| Change | (0.680) | (0.906) | (1.882) | (3.002) | (4.313) | (2.618) |

The treatment comparisons of 1 mg, 2 mg, and 3 mg doses of INT131 with placebo were statistically significant (p≤0.0109). This demonstrates that treatment with INT131 resulted in a statistically significant increase in adiponectin levels in patients suffering from a disease in which adiponectin levels are reduced (e.g. TD2). Thus, INT131 is therapeutically effective in treating patients with diseases (e.g. Huntington's disease) in which adiponectin levels are reduced.

Additionally, the treatment comparisons of 0.5 mg, 1 mg, and 3 mg doses of INT131 with pioglitazone 45 mg were statistically significant (p≤0.0408). Thus, the dose dependent increase of adiponectin levels by INT131 is independent from the increase resulting from pioglitazone.

### Conclusions

The effect of treatment on serum adiponectin was assessed, enabling a more direct comparison of the relative potencies of INT131 and pioglitazone 45 mg as selective PPARγ modulators. The mean change in adiponectin from baseline to Week 24 with LOCF (last observation carried forward) was 0.05 μg/mL for the placebo group, 0.56 μg/mL for the INT131 0.5 mg group, 1.28 μg/mL for the INT131 1 mg group, 3.27 μg/mL for the 2 mg group, 3.83 μg/mL for the INT131 3 mg group, and 2.96 μg/mL for the pioglitazone 45 mg group. Therefore, in a manner quantitatively different from the effects on HbA_{1c}, where the INT131 dose roughly equivalent to pioglitazone 45 mg is between 2 mg and 3 mg, a dose of INT131 between 1 mg and 2 mg was equivalent to pioglitazone 45 mg for increasing adiponectin levels.

Surprisingly, administration of INT131 at either 2 or 3 mg resulted in a greater upregulation of serum adiponectin levels than did administration of at least 22 times the amount of pioglitazone. Small amounts of INT131 are at least as efficacious in treating diseases in which adiponectin levels are reduced as are other drugs which also increase adiponectin levels. Since INT131 crosses the blood brain barrier more readily than other PPARγ agonists, less INT131 is required to achieve the same increase adiponectin, and INT131 has fewer side effects than other PPARγ agonists, INT131 is a superior treatment for neurological diseases.

Administration of 1, 2, or 3 mg of INT131 treats patients suffering from diseases in which adiponectin levels are reduced (e.g. Huntington's disease).

### Example 2: INT131 is a Potent Upregulator of Adiponectin in Healthy Subjects

### Method

A study was conducted to determine the effect of INT131 on serum adiponectin levels. Thirty healthy subjects were randomly selected to receive either placebo, 0.1 mg INT131, 1 mg INT131 or 4 mg INT131 daily for 14 days. To measure adiponectin levels blood was drawn at Days 1, 4, 8 and 14.

### Results

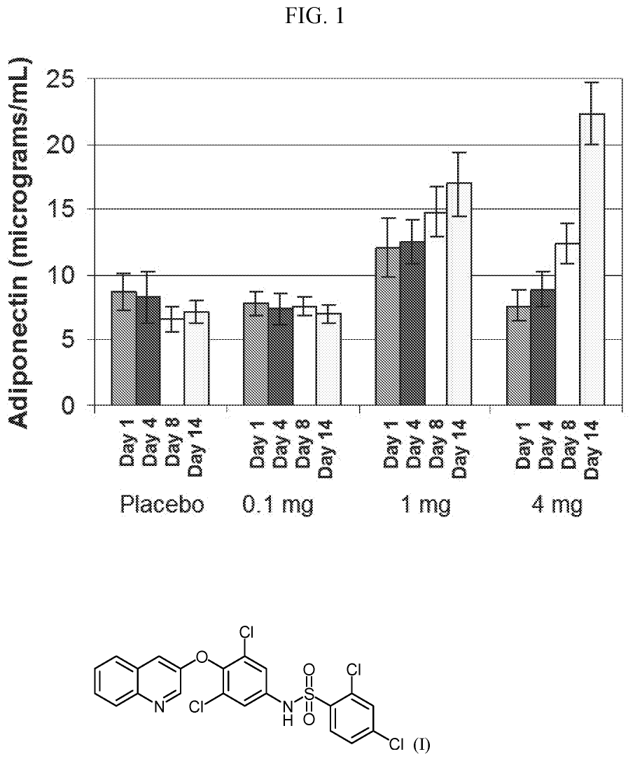

From Day 1 to Day 14 administration of placebo and 0.1 mg INT131 resulted in no significant change in serum adiponectin levels and further administration of 0.1 mg INT131 resulted in no significant change in adiponectin levels over placebo. See FIG. 1. However, administration of 1 mg or 4 mg INT131 resulted in a significant change in serum adiponectin levels over placebo and a significant change from Day 1 to Day 14. Thus, administration of INT131 is capable of upregulating adiponectin in healthy individuals.

Upregulation of adiponectin in subjects with Huntington's disease who have not developed signs and symptoms of the disease has a prophylactic effect. In subjects with Huntington's disease who have not developed signs and symptoms, administration of INT131 delay onset of the signs and symptoms.

### Example 3: INT131 Treats Huntington's Disease

INT-131 is evaluated in R6/2 Huntington's mice. R6/2 mice express mutant human exon 1 of the HTT gene. R6/2 mice develop Huntington phenotypes, which progress over time. These mice display motor and cognitive deficits, cortical cellular degeneration and striatal cellular morphological changes, as well as alterations in cortical and striatal synaptic transmission. In particular, R6/2 mice develop a loss of coordination, tremors, hypokinesis, abnormal gait, neuropathy, and premature death.

### INT-131 Compared to Placebo

After mice develop Huntington symptoms, the mice are administered INT-131 or a placebo at a regular dosing interval over a period of time. INT-131 doses include 1 mg/kg, 3 mg/kg, and 6 mg/kg. Huntington disease signs and symptoms are evaluated in each group over the period of time.

INT-131 slows progression and development of Huntington's disease in subjects administered INT-131. Mice administered INT-131 have reduced Huntington's disease symptoms.

### INT-131 Compared to Other Therapies

After mice develop Huntington symptoms, the mice are administered INT-131 or another therapy for treating Huntington's disease at a regular dosing interval over a period of time. INT-131 doses include 1 mg/kg, 3 mg/kg, and 6 mg/kg. Other therapies include those approved for treatment and those in development for treating Huntington's disease.

INT-131 is as effective, or more effective, than another therapy in slowing the progression or development of Huntington's disease. INT-131 is as effective, or more effective, than another therapy in treating the sign and symptoms of Huntington's disease.

What is claimed is:

1. A method of treating Huntington's disease in a subject in need thereof comprising administering to the subject a pharmaceutical composition comprising a therapeutically effective amount of a compound of formula (I), 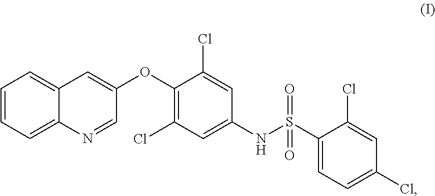 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

2. A method of treating the symptoms of Huntington's disease in a subject in need thereof comprising administering to the subject a pharmaceutical composition comprising a therapeutically effective amount of a compound of formula (I), 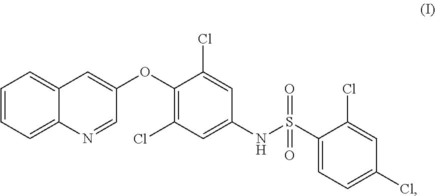 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

3. The method of claim 1, wherein the compound of formula (I), a pharmaceutically acceptable salt, prodrug, or isomer thereof is given prophylactically.

4. The method of claim 3, wherein onset of Huntington disease signs and symptoms are delayed.

5. The method of any one of claims 1 to 2, wherein the compound of formula (I) is in the form of a besylate salt.

6. The method of any one of claims 1 to 2, wherein the therapeutically effective amount is from about 0.1 to about 15 milligrams.

7. The method of claim 6, wherein the therapeutically effective amount is from about 1 to about 10 milligrams.

8. The method of claim 7, wherein the therapeutically effective amount is from about 2 to about 6 milligrams.

9. The method of claim 8, wherein the therapeutically effective amount is about 3 milligrams.

10. The method of any one of claims 1 to 2, wherein the pharmaceutical composition is administered to the subject twice a day, daily, every other day, three times a week, twice a week, weekly, every other week, twice a month, or monthly.

11. The method of claim 10, wherein the pharmaceutical composition is administered to the subject daily.

12. The method of any one of claims 1 to 2, wherein the pharmaceutical composition is administered to the subject daily and the therapeutically effective amount of the compound is about 3 milligrams.

13. The method of any one of claims 1 to 2, wherein the method provides an increase in adiponectin level in the subject by at least about 30%, at least about 68%, at least about 175%, or at least about 200%.

14. The method of claim 13, wherein the increase is by at least about 175%.

15. The method of any one of claims 1 to 2, wherein the method does not result in an increase in adipocytes or adipose tissue.

16. The method of any one of claims 1 to 2, wherein glucose metabolism in brain cells is increased.

17. The method of any one of claims 1 to 2, wherein glucose metabolism in adipose tissue is increased.

18. The method of any one of claims 1 to 2, wherein the lifespan of the subject is longer than the lifespan of a subject with Huntington's disease not administered the compound of formula (I), a pharmaceutically acceptable salt, prodrug, or isomer thereof.

19. The method of any one of claims 1 to 2, wherein the metabolic dysregulation in the subject is reduced.

20. The method of any one of claims 1 to 2, wherein the subject's appetite is increased.

21. The method of any one of claims 1 to 2, wherein the weight loss in the subject is reduced.

22. The method of any one of claims 1 to 2, wherein insulin resistance in the subject is reduced.

23. A method of treating Huntington's disease in a subject in need thereof comprising increasing adiponectin levels in the subject, wherein adiponectin levels are increased by administering to the subject a pharmaceutical composition comprising a therapeutically effective amount of a compound of formula (I), 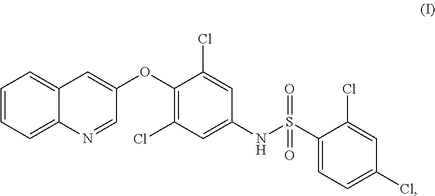 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

24. A method of treating the symptoms of Huntington's disease in a subject in need thereof comprising increasing adiponectin levels in the subject, wherein adiponectin levels are increased by administering to the subject a pharmaceutical composition comprising a therapeutically effective amount of a compound of formula (I), 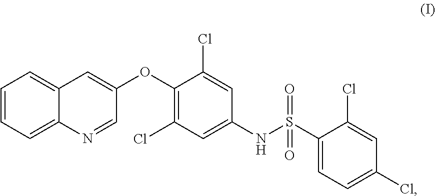 [SMILES: O=S(=O)(Nc1cc(Cl)c(Oc2cnc3ccccc3c2)c(Cl)c1)c1ccc(Cl)cc1Cl]
or a pharmaceutically acceptable salt, prodrug, or isomer thereof.

25. The method of claim 23, wherein the compound of formula (I), a pharmaceutically acceptable salt, prodrug, or isomer thereof is given prophylactically.

26. The method of claim 25, wherein onset of Huntington disease signs and symptoms are delayed.

27. The method of any one of claims 23 to 24, wherein the compound of formula (I) is in the form of a besylate salt.

28. The method of any one of claims 23 to 24, wherein the therapeutically effective amount is from about 0.1 to about 15 milligrams.

29. The method of claim 28, wherein the therapeutically effective amount is from about 1 to about 10 milligrams.

30. The method of claim 29, wherein the therapeutically effective amount is from about 2 to about 6 milligrams.

31. The method of claim 30, wherein the therapeutically effective amount is about 3 milligrams.

32. The method of any one of claims 23 to 24, wherein the pharmaceutical composition is administered to the subject twice a day, daily, every other day, three times a week, twice a week, weekly, every other week, twice a month, or monthly.

33. The method of claim 32, wherein the pharmaceutical composition is administered to the subject daily.

34. The method of any one of claims 23 to 24, wherein the pharmaceutical composition is administered to the subject daily and the therapeutically effective amount of the compound is about 3 milligrams.

35. The method of any one of claims 23 to 24, wherein the adiponectin level in the subject is increased by at least about 30%, at least about 68%, at least about 175%, or at least about 200%.

36. The method of claim 35, wherein the increase is by at least about 175%.

37. The method of any one of claims 23 to 24, wherein the method does not result in an increase in adipocytes or adipose tissue.

38. The method of any one of claims 23 to 24, wherein glucose metabolism in brain cells is increased.

39. The method of any one of claims 23 to 24, wherein glucose metabolism in adipose tissue is increased.

40. The method of any one of claims 23 to 24, wherein the lifespan of the subject is longer than the lifespan of a subject with Huntington's disease not administered the compound of formula (I), a pharmaceutically acceptable salt, prodrug, or isomer thereof.

41. The method of any one of claims 23 to 24, wherein the metabolic dysregulation in the subject is reduced.

42. The method of any one of claims 23 to 24, wherein the subject's appetite is increased.

43. The method of any one of claims 23 to 24, wherein the weight loss in the subject is reduced.

44. The method of any one of claims 23 to 24, wherein insulin resistance in the subject is reduced.
